(19) United States
(12) Patent Application Publication
(10) Pub. No.: US 2011/0064706 A1
(43) Pub. Date: Mar. 17, 2011

(54) METHOD OF PREVENTING, CONTROLLING AND AMELIORATING URINARY TRACT INFECTIONS AND SUPPORTING DIGESTIVE HEALTH BY USING A SYNERGISTIC CRANBERRY DERIVATIVE, A D-MANNOSE COMPOSITION AND A PROPRIETARY PROBIOTIC BLEND

(75) Inventors: John A. MINATELLI, Mt. Dora, FL (US); W. Stephen Hill, Ocala, FL (US); S. K. Dash, Edina, MN (US)

(73) Assignee: U.S. Nutraceuticals, LLC d/b/a Valensa International, Eustis, FL (US)

(21) Appl. No.: 12/889,450

(22) Filed: Sep. 24, 2010

Related U.S. Application Data
(63) Continuation-in-part of application No. 12/426,500, filed on Apr. 20, 2009.
Application No. 12/426,500 is a continuation-in-part of application No. 12/348,947, filed on Jan. 6, 2009.
(60) Provisional application No. 61/020,558, filed on Jan. 11, 2008.
(60) Provisional application No. 61/023,905, filed on Jan. 28, 2008.

(51) Int. Cl. A61K 35/74; A61P 13/02

(52) U.S. Cl. 424/93.45

(57) ABSTRACT

A method and composition prevents, controls and ameliorates urinary tract infections caused by E. coli by administering a therapeutically effective amount of a human dietary supplement composition comprising a cranberry derivative or proanthocyanidin containing concentrate, D-mannose and a proprietary probiotic blend. The cranberry derivative or proanthocyanidin containing concentrate comprises from about one percent (1.0%) by weight to about 95 percent (95.0%) by weight on a dry weight basis. The composition is formulated for delivering a D-mannose unit dosage between about 0.5 to about 5.0 grams per dose. A proprietary probiotic blend is included to provide 5 billion CFU per dose.

### RELATED APPLICATIONS

This application is a continuation-in-part application of U.S. patent application Ser. No. 12/426,500 filed Apr. 20, 2009, which is a continuation-in-part of U.S. patent application Ser. No. 12/348,947, filed Jan. 6, 2009, which is based upon U.S. provisional patent application Ser. No. 61/020,558 filed Jan. 11, 2008 and U.S. provisional patent application Ser. No. 61/023,905 filed Jan. 28, 2008.

### FIELD OF THE INVENTION

The present invention relates preventing, controlling and ameliorating urinary tract infections (UTI) using cranberry derivative, a D-Mannose composition and a proprietary probiotic blend.

### BACKGROUND OF THE INVENTION

Urinary tract infections (UT's) are generally defined as the presence (>100,000/mL) of bacteria in the urine. UTIs are commonly caused by Gram-negative bacteria, particularly Escherichia coli (E. coli), and infect primarily women. This infection is enabled by the adherence and colonization of bacteria to urinary tract epithelial cells. Adherence by E. coli is performed by proteinaceous fibers (fimbriae) on the bacteria cell wall, which attach to specific oligosaccharide receptors on uroepithelial cells. Antibiotics are commonly prescribed for treatment, but often promote bacterial resistance. One in four women also encounter recurrence of the infection and are often found to be prone to such infections. Natural substances which could treat and prevent UTIs could be useful for those suffering this condition since antibiotic treatment, in many cases causes, a secondary vaginal yeast infection requiring a subsequent antifungal treatment.

Consumption of cranberries has been found to be somewhat effective in addressing UTI infections. Cranberry products can prevent adhesion of certain bacteria fimbriae to uroepithelial cells in the urinary tract, thereby reducing the ability of the bacteria to create an infection (DiMartino et al., “Reduction of Escherichia Coli Adherence to Uroepithelial Bladder Cells After Consumption of Cranberry Juice; A Double-Blind Randomized Placebo-Controlled Cross-Over Trial,” World Journal of Urology, 2006); (Liu et al., “Role of Cranberry Juice on Molecular-Scale Surface Characteristics and Adhesion Behavior of Escherichia Coli,”, Biotechnology Bioengineering, 2006). Proanthocyanidins (condensed tannins) found in the cranberry juice have been shown to inhibit E. coli adherence (Howell et al., “Inhibition of the Adherence of P-Fimbriated Escherichia Coli to Uroepithelial-Cell Surfaces by Proanthocyanidin Extracts from Cranberries,” Journal of Medicine, 1998). Some E. coli fimbriae bind specifically to D-mannose and their binding unaffected by cranberry based ingredients including proanthocyanidins. D-Mannose is a simple monosaccharide (a simple sugar) with unusual characteristics. D-mannose, unlike sucrose or fructose, is metabolized very slowly in humans, therefore once consumed, D-mannose will enter the blood stream and quickly move to excretion via the kidneys followed by entry into the bladder in urine. D-mannose once in urine will cause the bacterial fimbriae sensitive to D-mannose binding to attach to the D-mannose, rather than epithelial cells. This allows the body to flush the D-mannose bound E. coli bacteria from the body. In addition, D-mannose can reverse epithelial bound E. coli competitively interrupting the initial phases of urinary tract infection. To mitigate existing UTIs and prevent recurrence, regular consumption of cranberry in combination with D-mannose and a proprietary probiotic blend will prevent bacteria from adherence, colonization and ultimately prevent an uncontrollable urinary tract infection. For this strategy to work, consumer compliance is necessary. D-mannose has a natural sweetness, cranberry juice and its derivatives generally possess acceptable flavors and the proprietary probiotic blend is GRAS (generally recognized as safe) and is free from corn, soy, wheat, sugar and contains no artificial colors and flavors.

Combinations as compositions using cranberry have been presented by others, for example, in GE2396811 (WO 2004/056380), the disclosure which is hereby incorporated by reference in its entirety. As noted in cited reference, described compositions include an extract from a plant that is a member of the Ericaceae, Rosaceae, Pinaceae or Vitaceae family and at least one sugar that is not metabolized or is only partly metabolized by the human or animal body. The sugar is preferably a monosaccharide such as L-arabinose, L-fucose, D-mannose, L-rhamnose, L-xylose, lyxose or galactose. A preferred composition includes an extract of cranberry with D-mannose and a proprietary probiotic blend. These compositions may be used to treat bacterial infection caused by E. coli, particularly urinary tract infections (UTIs). Compositions also include an anthocyanin, anthocyanidin or a proanthocyanidin and at least one sugar that is not metabolized or is only partly metabolized by the human or animal body are also described.

Examples of D-mannose compositions are also disclosed in U.S. Patent Publication Nos. 2007/0166292 and 2007/0244069; and U.S. Pat. Nos. 5,525,341 and 6,210,681, the disclosures of which are hereby incorporated by reference in their entirety.

Probiotic blend manufactured by UAS Laboratories, Inc, 9953 Valley View Road, Eden Prairie, Minn. 55344 USA, is a proprietary blend containing Lactobacillus acidophilus DDS-1 Superstrain, Bifidobacterium bifidum UABB-10; Bifidobacterium lactis UABLA-12; and Bifidobacterium longum UABL-14. The probiotic blend is fortified with fructooligosaccharide (FOS), a non-digestible carbohydrate to promote the growth of probiotics in the gastrointestinal tract. Lactobacillus acidophilus DDS-1 is a unique endogenous human strain extensively researched and protected by U.S. Trademark No. 1,685,959 and U.S. Pat. No. 3,689,640, the disclosure which is hereby incorporated by reference in its entirety.

Several studies have demonstrated the diverse beneficial effects of Lactobacillus acidophilus DDS-1 validating its use as a probiotic. L. acidophilus DDS-1 produces an antibacterial compound ‘acidophilin’ that demonstrated activity against Bacillus subtilis, Clostridium perfringens, Escherichia coli, Salmonella enteritidis, Salmonella typhimurium, Staphylococcus aureus and many other pathogenic bacterial strains (Shahani K M et al., “Natural antibiotic activity of L. acidophilus and bulgaricus. I. Cultural conditions for the production of antibiosis II. Isolation of acidophilin from L. acidophilus” Cult Dairy Prod J, 1976, 1977). Another study supports the role of L. acidophilus DDS-1 in the inhibition of the growth and adhesion of pathogens at the vaginal and urethral mucosa and shows positive effects in the prevention of urinary tract infection (Tramer J. “Inhibitory effect of L. acidophilus,” Nature 1966; 211:204-5), (Gerasimov SV. “Probiotic prophylaxis in pediatric recurrent urinary tract infection,” Clin Pediatr 2004; 43:95-8). Besides Lactobacillus acidophilus, Bifidobacterium species have demonstrated a protective effect against Escherichia coli O157:H7 in a mouse model (Shu et al., “A dietary probiotic (Bifidobacterium lactis) reduces the severity of Escherichia coli O157:H7 infection in mice”, Med. Microbiol. Immunol. 189:147-152).

The major attributes of probiotic blend towards human health are as follows. The probiotic strains are of human isolates, acid and bile resistant, help promote digestive health and immune function and create unfavorable environment for pathogens. As a result, the probiotic blend contributes to the good balance of the intestinal flora and vaginal health.

### SUMMARY OF THE INVENTION

Cranberry derivatives, D-mannose and proprietary probiotic blend are combined in a novel and unobvious concentration and proportion with various additives for preventing, controlling or ameliorating urinary tract infections caused by E. coli by administering a therapeutically effective amount of a human dietary supplement composition as a cranberry derivative or proanthocyanidin containing concentrate and D-mannose and proprietary probiotic blend combined in a form suitable for oral consumption. The cranberry derivative or proanthocyanidin containing concentrate may be formed from about one percent (1.0%) by weight to about 95 percent (95%) by weight on a dry weight basis and may be formulated for delivering a D-mannose unit dosage between about 0.5 to about 5.0 grams per dose.

A proprietary probiotic blend is added that comprises in one example at least one of Lactobacillus acidophilus DDS-1; Bifidobacterium bifidum UABB-10; Bifidobacterium lactic UABLA-12; and Bifidobacterium longum UABL-14 that competes and has activity against undesirable bacteria, including E-coli, and wherein the composition formed from the cranberry derivative or proanthocyanidin containing concentrate, D-mannose and a proprietary probiotic blend are effective together for binding to E-coli to aid in flushing the E-coli from the urinary tract system while preventing binding of E-coli to cells in the urinary tract system. The proprietary probiotic blend comprises a pH beneficial probiotic in one example that acidifies the vaginal mucosa. A diuretic may be added in one example, and in another example, an analgesic or antispasmodic or combination thereof.

In one aspect, the D-mannose is derived from a natural or synthetic source. The cranberry derivative can be derived from whole cranberries, juice, puree, extract, dried powder concentrate, seed extract, or any combination thereof. A carrier can also be administered for example, maltodextrin, starch, cellulose, food-grade silica or flow agents, or one or more acidulants, for example citric acid, malic acid or ascorbic acid. Acidulants are useful in lowering the pH of urine, a method known to reduce the susceptibility to infectious microbes.

In another aspect, the composition is prepared and packaged in a wet or dry form suitable for direct addition to water as a beverage pre-mix concentrate. Other food ingredient components can be added to increase the palatability of the formula, including, for example, natural and/or artificial flavors, nutritive and/or non-nutritive sweeteners, salts, acids or other suitable food ingredients. The composition can be incorporated into a solid or semi-solid food or a beverage. In one aspect, the composition is added to a liquid as a ready-to-drink beverage. The composition in another aspect can be provided as tablets, capsules, powders, emulsions, liquid concentrates, beverages, confectionery candies, including gummy bear confectioneries or chocolate, or encapsulated in liquid gels.

Other biologically active extracts and compounds can be added, including for example, vitamins, minerals, antioxidants, dietary fibers, tocopherols, tocotrienols, phytosterols, polysaccharides, polyphenolics and bioflavonoids. The composition can contain a naturally occurring diuretic including but not limited to, for example, saw palmetto, uva ursi, asparagus root, goldenrod, parsley, cleavers, dandelion, hydrangea, or any combination thereof. The composition can also contain a prescription diuretic, including but not limited to, for example, chlorothiazide, furosemide, theobromine, theophylline, oleandrin, or amiloride.

A capsule can contain the cranberry derivatives, D-mannose and a proprietary probiotic blend and other additives in an effective dosage form. The composition can be advantageously densified into a roller compacted powder to increase bulk density and decrease effective dose volume.

The proanthocyanidin containing concentrate ingredient of the invention can be derived from blueberry, grape, French maritime bark extract or any combination thereof either alone or as a juice concentrate bound to naturally occurring fibers, including but not limited to cranberry pomace derived from drying wet press cake from the juicing of fresh cranberries. In another aspect of the invention a proprietary probiotic blend containing Lactobacillus acidophilus DDS-1, Bifidobacterium bifidum UABB-10; Bifidobacterium lactis UABLA-12; and Bifidobacterium longum UABL-14 can be added that competes and has activity against undesirable bacteria, including E coli. Such probiotics promote good vaginal health by acidifying the vaginal area, producing anti-microbial hydrogen peroxide, competing for food with pathogenic microorganisms and adhering to epithelial cells of the vaginal mucosa leaving no space for invading pathogens. Golden seal (Hydrastis canadensis) can be added for increasing IgM production. Stinging nettle (Urtica dioica) can be added as a diuretic. Echinacea (Echinacea spp.) can be added for stimulating anti-inflammatory effects and immunomodulatory and immunostimulant activity and has been found effective for use in the treatment method and composition.

In order to ameliorate pain, an analgesic may also be added, for example, phenazopyridine hydrochloride, aspirin, phenacetin, or any of the NSAID class of analgesics or a narcotic such as codeine, butorphenol and the like or with antispasmotics to reduce the pain and discomfort associated with urinary tract infections.

### BRIEF DESCRIPTION OF THE DRAWINGS

Other objects, features, and advantages of the present invention will become apparent from the detailed description of the invention which follows when considered in light of the accompanying drawings in which:

FIG. 1 is a graph showing stability data for lactobacillus acidophilus.

FIG. 2 is a graph showing stability data for bifidobacterium bifidum.

FIG. 3 is a graph showing stability data for bifidobacterium longum.

FIG. 4 is a graph showing stability data for bifidobacterium lactis.

### DETAILED DESCRIPTION OF THE PREFERRED EMBODIMENTS

The present invention will now be described more fully hereinafter, in which preferred embodiments are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art.

D-Mannose is a simple sugar and related as a (steroisomer) to D-glucose. D-mannose is metabolized very slowly in humans allowing a steady state concentration in blood serum to carry the D-mannose to the kidneys for intact elimination in urine. The urinary tract infection occurs when E. coli enters the urethra and binds to the inner walls of the bladder, often reaching as far as the kidneys where a more dangerous infection can lead to kidney cell damage. The cell walls of the E. coli are covered with tiny finger like projections called fimbriae whose structures are important to cell wall recognition and subsequent infection forming a “glycoprotein”, also referred to as a “lectin”. D-Mannose competitively binds to the E. coli lectin binding recognition sites thus preventing attachment of uroepithelial cells. Bound E. coli can then be simply eliminated via urination. The inventors believe that a non-antibiotic method of eliminating E. coli from the urinary tract may be advantageous when considering antibiotic resistance and secondary yeast infections caused by antibiotic treatments. In addition antibiotics kill not only the unwanted micro-organisms but also kill friendly micro-organisms.

For example many females suffer from vaginal yeast infections following antibiotic use because the natural bacteria are killed along with the unwanted infectious bacteria and/or fungi, leaving antibiotic insensitive fungi, such as Candida albicans, to grow and reproduce further complicating the treatment to health. Long-term antibiotic use can lead to major disruptions in normal body microflora, disrupts health and is known to promote bacterial antibiotic resistance.

It has been found that the D-Mannose in combination with cranberry proanthocyanin concentrates and a proprietary probiotic blend can be helpful in these situations. In accordance with a non-limiting aspect of the present invention, the use of a cranberry derivative or proanthocyanidin containing concentrate in combination with D-Mannose and a proprietary probiotic blend and a diuretic and optionally other effective additives in a therapeutically effective amount with proper proportions is a suitable composition for oral consumption and complements the effect of D-Mannose since for example, E. coli populations contain both D-mannose sensitive and D-mannose insensitive binding and recognition sites. For example, the cranberry derivative or proanthocyanidin containing concentrate can be about one percent by weight to about 95 percent by weight on a dry weight basis and the total composition formulated for delivering a D-mannose unit dosage between 0.5 and 5.0 grams per dose. Although natural cranberry juice contains small amounts of D-Mannose the supplemental composition containing the cranberry derivative or proanthocyanidin containing concentrate or multiple cranberry derivative(s) and D-Mannose with a diuretic is effective and advantageous as a dietary supplement composition when each are proportioned in a specific manner.

In one non-limiting aspect, the cranberry derivative(s) are derived from whole cranberries, juice, puree, extract, dried powder concentrate, seed extract, or any combination thereof.

The composition is incorporated with a suitable carrier such as maltodextrin, starch, cellulose, food-grade silica, flow agents, and one or more acidulants such as citric acid, malic acid and ascorbic acid in a non-limiting example. The composition as a formula can be prepared and packaged in a wet or dry form suitable for direct addition to water to form a beverage drink. Other additives to the drink can also be used.

In one non-limiting and preferred aspect, the composition as a formulation delivers a D-mannose unit dosage between about 0.5 and about 5.0 grams per dose. As another non-limiting example, the cranberry derivative or proanthocyanidin containing concentrate typically comprises from about one percent (1.0%) by weight to about 95 percent (95.0%) by weight of the formula on a dry weight basis. This proportion is therapeutically effective and advantageous.

Other components can be added to increase the palatability of the formula, including for example, natural and/or artificial flavors, nutritive and/or non-nutritive sweeteners, salts, acids or other suitable food ingredients. The compositions can be incorporated into food and beverage, for example, as a human dietary supplement composition in a ready to drink beverage.

The compositions can also be in the form of tablets, capsules, powders, emulsions, liquid concentrates, beverages, confectionary candies and liquid gels. Other biologically active extracts and compounds can be added including for example, vitamins, minerals, antioxidants, dietary fiber, tocopherols, tocotrienols, phytosterols, polysaccharides, polyphenolics and bioflavonoids, analgesics or antispasmodics and have been found to add to the therapeutically effectiveness of the treatment method and composition.

The formula as a composition can contain a naturally occurring diuretic such as saw palmetto, uva ursi, asparagus root, goldenrod, parsley, cleavers, dandelion, hydrangea, and the like and extracts of such and in different combinations.

The formula as a composition can also contain a prescription diuretic such as chlorothiazide, furosemide, theobromine, theophylline, oleandrin, amiloride and analgesic or an antispasmotic as previously described.

An analgesic or antispasmodic can be added such as scopolamine, phenazopyridine hydrochloride, aspirin, acetaminophen and any of the class of NSAIDS or a herbal such as scopolia root, angelica root and the like to reduce the pain of E. coli urinary tract infections.

Additionally, the formulation may be compacted in suitable roller compaction device in order to increase the bulk density, thereby reducing the consumption volume needed for an effective dose. It is known that consumer dose compliance decreases with increasing capsule size and number, therefore formulation compaction can increase consumer compliance and resulting effectiveness, particularly in capsule presentations. For example, the composition can have a higher density, resulting in an effective therapeutic dosage using two capsules instead of four capsules when capsules are used in the method of treatment.

The proanthocyanidin containing concentrate can be derived from blueberry, grape, French maritime bark extract and D-mannose or any combination thereof. In another aspect a proprietary probiotic blend containing Lactobacillus acidophilus DDS-1, Bifidobacterium bifidum UABB-10; Bifidobacterium lactis UABLA-12; and Bifidobacterium longum UABL-14 can be added that competes and has activity against undesirable bacteria, including E coli. The probiotic blend promotes good vaginal health by acidifying the vaginal area, producing anti-microbial hydrogen peroxide, competing for food with pathogenic microorganisms and adhering to epithelial cells of the vaginal mucosa leaving no space for invading pathogens.

Preferred strains of probiotics as stated earlier include: Lactobacillus acidophilus DDS-1; Bifidobacterium bifidum UABB-10; Bifidobacterium lactis UABLA-12; and Bifidobacterium longum UABL-14.

As is known, probiotics are microorganisms that are beneficial for the host, and more particularly, the human host. They provide measurable health benefits to the digestive tract and interact with other bacteria in the intestine and other sections of the alimentary canal.

These probiotics promote better digestion and support production of proteases (protein digesting enzymes) and lipases (fat-digesting enzymes), and creation of essential vitamins. In the GI (gastrointestinal) tract, probiotics manufacture several B vitamins and vitamin K. These probiotics also can balance intestinal bacteria after antibiotic therapy. Antibiotics disrupt probiotic populations in the lower GI tract, potentially causing unpleasant side effects during and after therapy. Studies show that the large intestinal ‘ecosystem’ returns to its pre-antibiotic balance more quickly following antibiotic therapy when probiotics are administered.

There is some competitive inhibition of harmful or ‘bad’ bacteria using probiotics. ‘Good’ bacteria (probiotics) compete with ‘bad’ bacteria for fuel and space, making it difficult for harmful bacteria to gain presence in the GI tract. Some probiotics produce natural antibiotic substances (like acidophilin, in the case of L. acidophilus DDS-1) that directly attack harmful bacteria, including some of the most feared food-borne pathogens. Thus, today's growing problem of antibiotic-resistant bacteria makes the need for alternatives all the more urgent.

There is also a balancing of the immune response. Probiotics impact the immune system, stimulating the production of immunoglobulins (antibodies) and cytokines (chemicals made in the body that modulate inflammation) and promote greater resistance to infection and to inflammatory disorders of the GI tract such as Crohn's disease and ulcerative colitis.

There is also a direct effect on the genes of some pathogens. Probiotics appear to have the capacity to affect the expression of those genes in ways that reduce pathogens' virulence. (For example, see Corr S C, Hill C, Gahan C G, “Chapter 1: Understanding the Mechanisms by Which Probiotics Inhibit Gastrointestinal Pathogens,” Adv Food Nutr Res 2009; 56:1-15.)

Probiotic products are now available in different formulations with Lactobacillus acidophilus, Bifidobacterium bifidum, Bifidobacterium longum, Bifidobacterium lactis and others with or without Prebiotic Fructooligosaccharide (FOS). The most common probiotic products are 1) Lactobacillus acidophilus DDS-1 and Bifidobacterium longum UABL-14 with FOS; 2) Lactobacillus acidophilus DDS-1 and Bifidobacterium lactis UABLA-12 with FOS; 3) Lactobacillus acidophilus DDS-1, Bifidobacterium bifidum UABB-10; Bifidobacterium lactis UABLA-12; and Bifidobacterium longum UABL-14 with FOS and 4) lactobacillus acidophilus DDS-1 with FOS.

There are major benefits that probiotic products offer: 1) digest foods and alleviate digestive disorders; 2) enhance synthesis of B vitamins and improve absorption of calcium; 3) keep E. coli in check; 4) promote vagina health and keep yeast in check; 5) improve immune function; 6) support the management of acne; and 7) help maintain normal cholesterol.

Research shows that seventy percent of women and forty percent of men have yeast infections to some degree as a result of heavy use of antibiotics, cortisone's and poor eating habits. Studies at Minneapolis V.A. Hospital show the average persons in their current life style, hardly have any friendly bacteria in their intestines. Supplementation of certain probiotics shows the presence of these probiotics in the intestine in great numbers. It is apparent that a polluted environment, processed food, chlorinated water and heavy use of antibiotics and other medications can destroy the friendly microflora (probiotics), thus making the body susceptible to yeast infection and other diseases.

Those familiar with the benefits of a probiotic often consume yoghurt, a long-recognized source of friendly bacteria. Unfortunately, commercial yoghurt often lack the probiotic. Even the commercial yoghurt fortified with Lactobacillus acidophilus and sitting on the shelf for several days does not have viable probiotics as they tend to produce bacteria-killing acids. For this reason, fresh, homemade yoghurt can be a reliable source of probiotic(s). However, it often fails to provide sufficient amounts for its intended purpose. As yoghurt and other foods do not supply the needed probiotics, supplementation of probiotics on a daily basis is highly recommended. Probiotic capsules, tablets or powder supplying 2 to 5 billion live organisms per gram (CFU/g) should be taken daily for maintenance and higher quantities are recommended following antibiotic therapy or under conditions of yeast infection, ulcer, cholesterol, digestive disorders and acne.

All probiotic products are not the same. The name Acidophilus or probiotic does not mean anything unless the probiotic product: 1) contains the right strain(s); 2) is viable; 3) remains stable and viable for a long period; 4) has ability to survive in the intestine; and 5) produces beneficial effects in the intestine.

Probiotic products sitting on the shelf (without refrigeration) lose potency fast. For this reason, refrigerated, viable probiotic products are recommended.

Non-dairy probiotic products are recommended by health professionals for persons allergic to dairy products and for persons with yeast infection.

Nitrogen-flushed packaging of probiotics enhances the keeping quality of the probiotics.

Fortification of probiotics with prebiotic Fructooligosaccharides (FOS) enhances the value of probiotics selectively and enhances their growth in the gut. Combinations of unknown and undesirable bacteria in probiotic products can even be harmful to the human body. Also, combinations of known and friendly bacteria in a disproportionate manner can alter flora balance in the gut. A more detailed explanation of probiotics that can be used is now set forth. These probiotics can be obtained from UAS Laboratories, Inc.

Product Name: Lactobacillus acidophilus
Ingredients: Lactobacillus acidophilus and Rice maltodextrin.
Manufacturing: Lactobacillus acidophilus is manufactured through a fermentation process and freeze-dried. The culture is blended with Rice maltodextrin. The culture is tested negative for salmonella and other pathogens.
Usage: To supplement the diet in maintaining normal intestinal flora.
Potency: 10 billion (10×109 CFU/g). Also available in 15 billion (15×10⁹ CFU/g) and 200 billion (200×109 CFU/g).
Packaging: 1 Kg, 5 Kg and 20 Kg packets/pouches.
Storage Requirements Store below 40° F. (4° C.) and at low relative humidity.
Shelf Life: Stable for 24 months at room temperature, however, refrigeration is recommended.

### Analytical Data:

a. Appearance: White, odorless powder.
b. Lot No:
c. Formulation: Lactobacillus acidophilus is blended with Rice maltodextrin. Assay more than 10×109 CFU/g.
d. Other Tests:

i. Coliform: Negative

ii. Salmonella: Negative

iii. Listeria: Negative

iv. E. Coli: Negative

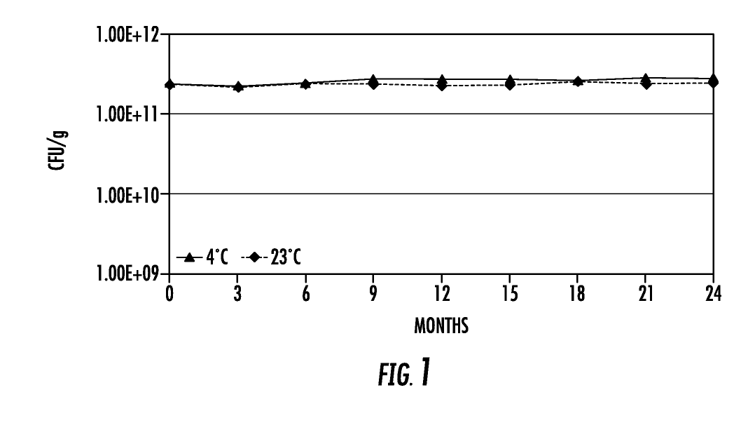

Stability data is shown in FIG. 1

### Allergen Statement:

| | | | |
|---|---|---|---|
| | | Free | |
| Item | Amount | From | Details |

| | |
|---|---|
| GMO Derived Material | Yes |
| Bovine Spongoform | Yes |
| Encephalopathy | |
| Animal Derived Material | Yes |
| Dairy Ingredient(s), | Yes |
| Product(s) | |
| Nuts e.g Peanuts | Yes |
| Egg | Yes |
| Artificial colourings | Yes |
| Aspartame | Yes |
| Saccharin | Yes |
| Sucralose | Yes |
| Tartrazine | Yes |
| Other artificial | Yes |
| sweetener(s) | |
| Dextrin | Yes |
| Starch | Yes |
| Gluten | Yes |
| Sorbitol | Yes |
| Corn | Yes |
| Wheat | Yes |
| Yeast | Yes |
| Gluten | Yes |
| Soyabean | Yes |
| Propyl Hydroxybenzoate | Yes |
| Methyl Hydroxybenzoate | Yes |
| Ethyl Hydroxybenzoate | Yes |
| Sodium Propyl | Yes |
| Hydroxybenzoate | |
| Sodium Methyl | Yes |
| Hydroxybenzoate | |
| Sodium Ethyl Hydroxybenzoate | Yes |
| Sodium Benzoate | Yes |
| Calcium Benzoate | Yes |
| Potassium Benzoate | Yes |
| Benzoic Acid | Yes |
| Sodium Sulfite | Yes |
| Sodium Metabisulfite | Yes |
| Potassium Metabisulfite | Yes |
| Other preservative(s) | Yes |
| Antioxidents | Yes |
| Sodium Chloride | Yes |
| Royal jelly | Yes |
| Ethanol | Yes |
| Flavour/Flavourings | Yes |
| Suitable for Vegetarians | Yes |

Product Name: Bifidobacterium bifidum
Ingredients: Bifidobacterium bifidum and Rice maltodextrin
Manufacturing: Bifidobacterium bifidum is manufactured through a fermentation process and freeze-dried. The culture is blended with Rice maltodextrin. The culture is tested negative for salmonella and other pathogens.
Usage: To supplement the diet in maintaining normal intestinal flora.
Potency: 10 billion (10×109 CFU/g). Also available in 15 billion (15×109 CFU/g) and 500 billion (500×109 CFU/g).
Packaging: 5 Kg packets/pouches.
Storage Requirements Store below 40° F. (4° C.) and at low relative humidity.
Shelf Life: Stable for 24 months at room temperature, however, refrigeration is recommended

### Analytical Data:

a. Appearance: White, odorless powder.
b. Lot No:
c. Formulation: Bifidobacterium bifidum is blended with Rice maltodextrin. Assay more than 10×109 CFU/g.
d. Other Tests:

i. Coliform: Negative

ii. Salmonella: Negative

iii. Listeria: Negative

iv. B. Coli: Negative

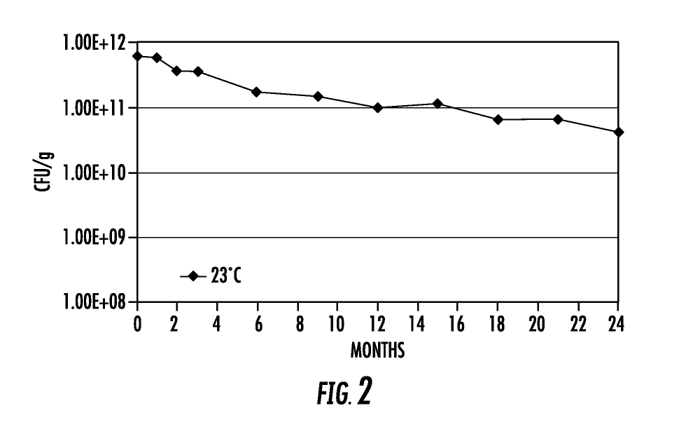

Stability Data is shown in FIG. 2

### Allergen Statement;

| | | | |
|---|---|---|---|
| | | Free | |
| Item | Amount | From | Details |

| | |
|---|---|
| GMO Derived Material | Yes |
| Bovine Spongoform | Yes |
| Encephalopathy | |
| Animal Derived Material | Yes |
| Dairy Ingredient(s), | Yes |
| Product(s) | |
| Nuts e.g Peanuts | Yes |
| Egg | Yes |
| Artificial colourings | Yes |
| Aspartame | Yes |
| Saccharin | Yes |
| Sucralose | Yes |
| Tartrazine | Yes |
| Other artificial | Yes |
| sweetener(s) | |
| Dextrin | Yes |
| Starch | Yes |
| Gluten | Yes |
| Sorbitol | Yes |
| Corn | Yes |
| Wheat | Yes |
| Yeast | Yes |
| Gluten | Yes |
| Soyabean | Yes |
| Propyl Hydroxybenzoate | Yes |
| Methyl Hydroxybenzoate | Yes |
| Ethyl Hydroxybenzoate | Yes |
| Sodium Propyl | Yes |
| Hydroxybenzoate | |
| Sodium Methyl | Yes |
| Hydroxybenzoate | |
| Sodium Ethyl Hydroxybenzoate | Yes |
| Sodium Benzoate | Yes |
| Calcium Benzoate | Yes |
| Potassium Benzoate | Yes |
| Benzoic Acid | Yes |
| Sodium Sulfite | Yes |
| Sodium Metabisulfite | Yes |
| Potassium Metabisulfite | Yes |
| Other preservative(s) | Yes |
| Antioxidents | Yes |
| Sodium Chloride | Yes |
| Royal jelly | Yes |
| Ethanol | Yes |
| Flavour/Flavourings | Yes |
| Suitable for Vegetarians | Yes |

Product Name: Bifidobacterium longum
Ingredients: Bifidobacterium longum and Rice maltodextrin
Manufacturing: Bifidobacterium longum is manufactured through a fermentation process and freeze-dried. The culture is blended with Rice maltodextrin. The culture is tested negative for salmonella and other pathogens.
Usage: To supplement the diet in maintaining normal intestinal flora.
Potency: 10 billion (10×109 CFU/g). Also available in 15 billion (15×109 CFU/g) and 75 billion (75×109 CFU/g),
Packaging: 1 Kg and 5 Kg packets/pouches.
Storage Requirements Store below 40° F. (4° C.) and at low relative humidity.
Shelf Life: Stable for 24 months at room temperature, however, refrigeration is recommended.

### Analytical Data:

a. Appearance: White, odorless powder.
b. Lot No:
c. Formulation: Bifidobacterium longum is blended with rice starch. Assay more than 10×109 CFU/g.
d. Other Tests:

i. Coliform: Negative

ii. Salmonella: Negative

iii. Listeria: Negative

iv. E. Coli: Negative

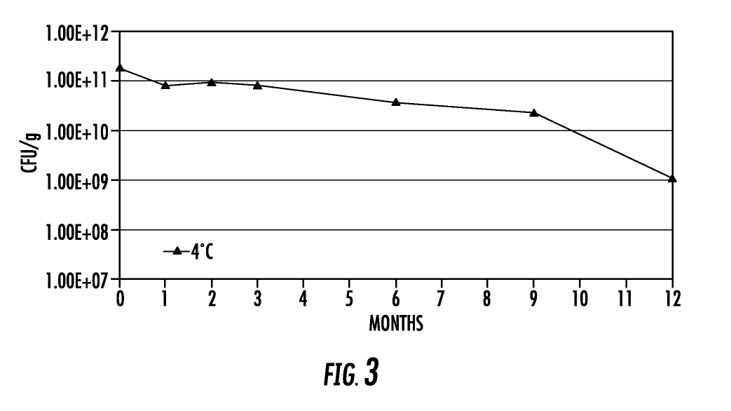

Stability Data is shown in FIG. 3

### Allergen Statement:

| | | | |
|---|---|---|---|
| | | Free | |
| Item | Amount | From | Details |

| | |
|---|---|
| GMO Derived Material | Yes |
| Bovine Spongoform | Yes |
| Encephalopathy | |
| Animal Derived Material | Yes |
| Dairy Ingredient(s), | Yes |
| Product(s) | |
| Nuts e.g Peanuts | Yes |
| Egg | Yes |
| Artificial colourings | Yes |
| Aspartame | Yes |
| Saccharin | Yes |
| Sucralose | Yes |
| Tartrazine | Yes |
| Other artificial | Yes |
| sweetener(s) | |
| Dextrin | Yes |
| Starch | Yes |
| Gluten | Yes |
| Sorbitol | Yes |
| Corn | Yes |
| Wheat | Yes |
| Yeast | Yes |
| Gluten | Yes |
| Soyabean | Yes |
| Propyl Hydroxybenzoate | Yes |
| Methyl Hydroxybenzoate | Yes |
| Ethyl Hydroxybenzoate | Yes |
| Sodium Propyl | Yes |
| Hydroxybenzoate | |
| Sodium Methyl | Yes |
| Hydroxybenzoate | |
| Sodium Ethyl Hydroxybenzoate | Yes |
| Sodium Benzoate | Yes |
| Calcium Benzoate | Yes |
| Potassium Benzoate | Yes |
| Benzoic Acid | Yes |
| Sodium Sulfite | Yes |
| Sodium Metabisulfite | Yes |
| Potassium Metabisulfite | Yes |
| Other preservative(s) | Yes |
| Antioxidents | Yes |
| Sodium Chloride | Yes |
| Royal jelly | Yes |
| Ethanol | Yes |
| Flavour/Flavourings | Yes |
| Suitable for Vegetarians | Yes |

Product Name: Bifidobacterium lactis
Ingredients: Bifidobacterium lactis and Rice maltodextrin
Manufacturing: Bifidobacterium lactis is manufactured through a fermentation process and freeze-dried. The culture is blended with Rice maltodextrin. The culture is tested negative for salmonella and other pathogens.
Usage: To supplement the diet in maintaining normal intestinal flora.
Potency: 10 billion (10×109 CFU/g). Also available in 15 billion (15×109 CFU/g) and 500 billion (500×109 CFU/g).
Packaging: 1 Kg and 5 Kg packets/pouches.
Storage Requirements Store below 40° F. (4° C.) and at low relative humidity.
Shelf Life: Stable for 24 months at room temperature, however, refrigeration is recommended.

### Analtical Data:

a. Appearance: White, odorless powder.
b. Lot No:
c. Formulation: Bifidobacterium lactis is blended with rice starch. Assay more than 10×109 CFU/g.
d. Other Tests:

i. Coliform: Negative

ii. Salmonella: Negative

iii. Listeria: Negative

iv. E. Coli: Negative

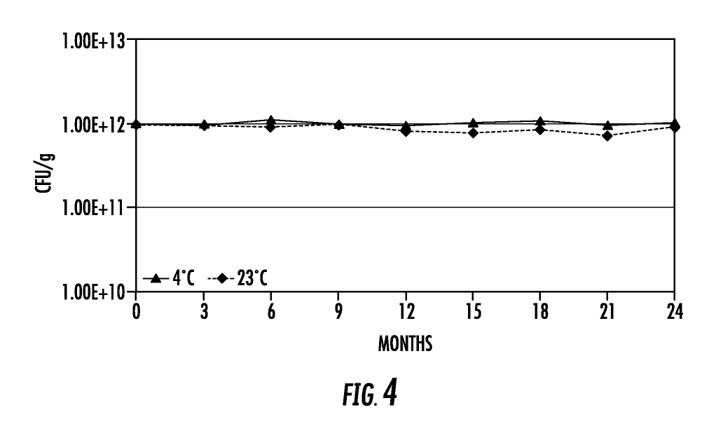

Stability Data is shown in FIG. 4

### Allergen Statement:

| | | | |
|---|---|---|---|
| | | Free | |
| Item | Amount | From | Details |

| | |
|---|---|
| GMO Derived Material | Yes |
| Bovine Spongoform | Yes |
| Encephalopathy | |
| Animal Derived Material | Yes |
| Dairy Ingredient(s), | Yes |
| Product(s) | |
| Nuts e.g Peanuts | Yes |
| Egg | Yes |
| Artificial colourings | Yes |
| Aspartame | Yes |
| Saccharin | Yes |
| Sucralose | Yes |
| Tartrazine | Yes |
| Other artificial | Yes |
| sweetener(s) | |
| Dextrin | Yes |
| Starch | Yes |
| Gluten | Yes |
| Sorbitol | Yes |
| Corn | Yes |
| Wheat | Yes |
| Yeast | Yes |
| Gluten | Yes |
| Soyabean | Yes |
| Propyl Hydroxybenzoate | Yes |
| Methyl Hydroxybenzoate | Yes |
| Ethyl Hydroxybenzoate | Yes |
| Sodium Propyl | Yes |
| Hydroxybenzoate | |
| Sodium Methyl | Yes |
| Hydroxybenzoate | |
| Sodium Ethyl Hydroxybenzoate | Yes |
| Sodium Benzoate | Yes |
| Calcium Benzoate | Yes |
| Potassium Benzoate | Yes |
| Benzoic Acid | Yes |
| Sodium Sulfite | Yes |
| Sodium Metabisulfite | Yes |
| Potassium Metabisulfite | Yes |
| Other preservative(s) | Yes |
| Antioxidents | Yes |
| Sodium Chloride | Yes |
| Royal jelly | Yes |
| Ethanol | Yes |
| Flavour/Flavourings | Yes |
| Suitable for Vegetarians | Yes |

Stinging nettle (Urtica dioica) can be added as a diuretic. Echinacea (echinecea spp.) can be added for stimulating anti-inflammatory effects and immunomodulatory and immunostimulant activity.

As noted above, goldenseal acts to increase IgM and similar components include Mahonia (Oregon grape) and Berberis (Barberry). It is believed that these components have the ability to inhibit drug resistance efflux pumps (MDR pumps) of bacteria. Goldenseal contains isoquinoline alkaloids: hydrastine, berberin, berberastine, hydrastinine, tetrahydroberberastine, canadine, and canalidine. Possibly the action of 8-oxotetrahydrothalifenine is operative Berberine and hydrastine are believed to act as quaternary bases.

Proanthocyanidins as identified above can also be found in many plants, for example, apples, pine bark, cinnamon, grape seed, cocoa, grape skin, and red wines of Vitis vinifera. Bilberry, cranberry, black currant, green tea, black tea and other plants also contain these flavonoids. The berries of chokeberry, such as black chokeberry, have high concentrations of proanthocyanidin and can be used.

Proanthocyanidins are known to bind to p-pilitated E. coli which are insensitive to binding by D-mannose. They can act also as vasoactive polyphenols such as in red wine and reduce the risk of coronary heart disease. They are believed to suppress production of a protein endothelin-1 that constricts blood vessels. Proanthocyanidins are believed to have antioxidant activity and stabalize collagen maintenance of elastin—two critical proteins in connective tissue that support organs, joints, blood vessels, and muscle. Proanthocyanidins are also believed to reduce histamine production and beneficial in treating allergies while also improving circulation by strengthening capillary walls. They are also believed to inhibit enzymes that break down collagen and help collagen repair and act as a sunscreen. Proanthocyanidins can cross the blood-brain barrier to fight free radicals in the vessels of the brain and increase mental acuity.

As to the diuretic action of stinging nettle, some studies show that a sex hormone binding globulin (SHBG), aromatase, epidermal growth factor and prostate steroid membrane receptors are involved in the anti-prostatic effect. It is not clear that 5α-reductase or androgen receptors are used. Some anti-inflammatory activity may be affected by extract and a polysaccharide fraction. It is believed also to contain different acids, for example, silicic, threonic, and formic acids and contain some amines such acetylcholine, choline, serotonin and histamine as well as flavonoids. A polysaccharide fraction could also aid in an anti-inflammatory effect and polypeptide fraction. Hops contain alpha- and beta-acids, where the alpha-acids occur as humulone, cohumuline and adhumulone, and essential oils and prenylfiavonoids.

Hops can have a sedative effect and also act as a potent estrogen receptor agonist in estrogen-responsive cells and aid in treating menstrual symptoms. Bitter acids in hops have an antibacterial and antifungal activity. Marshmallow root is believed to have bactericidal and anti-inflammatory properties. Myrhh is typically found as an oleo-gum and includes a volatile oil of sesquiterpenes, sterols, and steroids and can be used for antiparasitic effects and infections for females and males. It is believed that honey bee pollen contains some Apalbumin1 (Apa1) as a royal jelly (RJ) and honey glycoprotein having various biological properties. It could possibly stimulate macrophages to release tumor necrosis factor alpha (TNFalpha) and possibly has immuno-stimulatory effects. Plantain leaf is useful in GI therapy and treats hyperlipidemia through various actions. It includes various acids with various flavonoids.

Oregon Grape has various alkaloids, including berberine, berbamine, canadine, and hydrastine and can treat diarrhea in patients infected with E. coli, such as by slowing the transit time in the intestine and possibly inhibiting the ability of bacteria to attach to human cells, which helps prevent infections in the intestines and urinary tract. Echinacea has antibacterial properties possibly selectively modulates the catalytic activity of CYP3A at hepatic and intestinal sites.

Many modifications and other embodiments of the invention will come to the mind of one skilled in the art having the benefit of the teachings presented in the foregoing description. Therefore, it is understood that the invention is not to be limited to the specific embodiments disclosed, and that modifications and embodiments are intended to be included within the scope of the appended claims.

That which is claimed is:

1. A method of preventing, controlling and ameliorating urinary tract infections caused by E-coli by administering a therapeutically effective amount of a human dietary supplement composition comprising a cranberry derivative or proanthocyandin containing concentrate and D-mannose combined in a form suitable for oral consumption, and comprising a proprietary probiotic blend comprising at least one of Lactobacillus acidophilus DDS-1, Bifidobacterium bifidum UABB-10, Bifidobacterium lactis UABLA-12, and Bifidobacterium longum UABL-14 that compete and has activity against undesirable bacteria, including B coli and wherein the composition formed from the cranberry derivative or proanthocyandin containing concentrate, D-mannose, and a proprietary probiotic blend are effective together for binding to E-coli to aid in flushing the E-coli from the urinary tract system while preventing binding of E-coli to cells in the urinary tract system.

2. The method according to claim 1, wherein said probiotic comprises a pH beneficial probiotic that acidifies the vaginal mucosa.

3. The method according to claim 1, wherein the cranberry derivative or proanthocyandin containing concentrate comprises from about one percent (1.0) by weight to about 95 percent (95.0) by weight on a dry weight basis.

4. The method according to claim 1, wherein said composition is formulated for delivering a D-mannose unit dosage between about 0.5 to about 5.0 grams per dose.

5. The method according to claim 1, and further comprising a diuretic.

6. The method according to claim 1, and further comprising an analgesic or antispasmodic or combination thereof.

7. A human dietary supplement composition effective to prevent, control and ameliorate urinary tract infections caused by E-coli and comprising a cranberry derivative or proanthocyandin containing concentrate and D-mannose combined in a form suitable for oral consumption, and further comprising a proprietary probiotic blend comprising at least one of Lactobacillus acidophilus DDS-1, Bifidobacterium bifidum UABB-10, Bifidobacterium lactis UABLA-12, and Bifidobacterium longum UABL-14 that compete and has activity against undesirable bacteria, including E coli and wherein the composition formed from the cranberry derivative or proanthocyandin containing concentrate, D-mannose, and probiotic are effective together for binding to E-coli to aid in flushing the E-coli from the urinary tract system while preventing binding of E-coli to cells in the urinary tract system.

8. The composition according to claim 7, wherein said probiotic comprises a pH beneficial probiotic that acidifies the vaginal mucosa.

9. The composition according to claim 7, wherein the cranberry derivative or proanthocyandin containing concentrate comprises from about one percent (1.0%) by weight to about 95 percent (95.0%) by weight on a dry weight basis.

10. The composition according to claim 7, wherein said composition is formulated for delivering a D-mannose unit dosage between about 0.5 to about 5.0 grams per dose.

11. The composition according to claim 7, and further comprising a diuretic.

12. The composition according to claim 7, and further comprising an analgesic or antispasmodic or combination thereof.
